(19) United States
(12) Patent Application Publication
(10) Pub. No.: US 2024/0293537 A1
(43) Pub. Date: Sep. 5, 2024

(54) AMPHIPHILIC IMIQUIMOD-GRAFTED LAURYL GAMMA-POLYGLUTAMATE AND USE THEREOF

(71) Applicant: JIANGSU ACADEMY OF AGRICULTURAL SCIENCES, Nanjing (CN)

(72) Inventors: Wenzhu YIN, Nanjing (CN); Jinqiu ZHANG, Nanjing (CN); Mingxu ZHOU, Nanjing (CN); Yu LU, Nanjing (CN)

(21) Appl. No.: 17/800,066

(22) PCT Filed: Nov. 17, 2021
(86) PCT No.: PCT/CN2021/131111

§ 371 Date: Aug. 16, 2022

(30) Foreign Application Priority Data
Oct. 12, 2021 (CN) 202111186210.0

(51) Int. Cl. A61K 39/39; A61K 39/00; A61K 47/59; A61K 49/00; C08G 69/48

(52) U.S. Cl. CPC A61K 39/39; A61K 47/595; A61K 49/0043; C08G 69/48; A61K 2039/55511

(57) ABSTRACT

An amphiphilic imiquimod-grafted lauryl γ-polyglutamate is prepared by adopting a method comprising the following steps: (1) dispersing γ-polyglutamic acid in an aprotic solvent in an anhydrous atmosphere, then adding a catalyst N,N-dimethylformamide, adding dropwise a chlorinating agent under stirring, and keep reaction of the mixture for 10-40 hours; (2) adding imiquimod, liposoluble alcohol and an acid-binding agent into the solution obtained in the step (1) after reaction, and reacting for 42-54 hours; and (3) after purification, obtaining an amphiphilic imiquimod-grafted lauryl γ-polyglutamate material.

### TECHNICAL FIELD

The present invention belongs to the field of biomedical materials, and particularly relates to amphiphilic imiquimod-grafted lauryl 7-polyglutamate and use thereof.

### BACKGROUND

Vaccination is considered one of the most economical, convenient and effective methods for preventing infectious diseases in today's society. Antigen delivery is a crucial step in the immunization process. In the practical application of vaccines, since many antigens are not sufficient to confer an immune antibody response when administered alone, it is necessary to design adjuvants capable of immobilizing the antigens and stimulating the immune response.

Toll-like receptors (TLRs) are pattern recognition receptors (PRRs), and such the receptors, when activated by microorganism-specific conserved product-pathogen-associated molecular patterns (PAMPs), can not only induce innate immune responses, but also activate the acquired immune system, and thus are theoretically ideal choices for adjuvants. In recent years, synthetic imiquimod (R837), as a TLR 7 agonist, is a small molecule immunomodulator having excellent antiviral and antitumor properties. Because of its small relative molecular weight, the imiquimod can enter a body through various ways, the activity of the antigen presenting cells is improved, many dendritic cells, macrophages, B cells, T cells and the like are gathered to a vaccination site, and the local immune response is enhanced. However, imiquimod also has the following drawbacks: (1) the solubility in water and common organic solvents is low, so that it is not easy for imiquimod to prepare as an injection, and imiquimod has certain toxicity to cells; (2) the single use of R837 may cause some adverse reactions, such as erythema, erosion, desquamation/exfoliation and edema, which are common adverse reactions; (3) the pharmacokinetics of imiquimod are characterized by rapid diffusion from local (e.g., subcutaneous or intramuscular) to the whole body, resulting in unwanted intrinsic immune activation in multiple distal tissues. Due to these drawbacks, the use of imiquimod is limited.

### SUMMARY

An objective of the present invention is to provide amphiphilic imiquimod-grafted lauryl γ-polyglutamate, which has good water solubility, good biocompatibility and reduced toxic and side effects, and can improve the specific immune response of organisms, and thus is an ideal adjuvant.

Another objective of the present invention is to provide use of amphiphilic imiquimod-grafted lauryl γ-polyglutamate in a vaccine adjuvant.

The objectives of the present invention is implemented by adopting the following technical solution:

Provided is amphiphilic imiquimod-grafted lauryl γ-polyglutamate, which is prepared by adopting a method comprising the following steps:
(1) dispersing γ-polyglutamic acid in an aprotic solvent in an anhydrous atmosphere, then adding a catalyst N,N-dimethylformamide, adding dropwise a chlorinating agent under stirring, and reacting the mixture for 10-40 hours;
(2) adding imiquimod, liposoluble alcohol and an acid-binding agent into the solution obtained in the step (1) after reaction, and reacting for 42-54 hours; and
(3) after purification, obtaining an amphiphilic imiquimod-grafted lauryl 7-polyglutamate material.

In the present invention, a molecular weight of γ-polyglutamic acid is 10,000-2,000,000, and preferably 300,000-700,000; the chlorinating agent is thionyl chloride, oxalyl chloride or phosphorus pentachloride, and preferably thionyl chloride or oxalyl chloride; the liposoluble alcohol is C8-C24 alcohol, alicyclic alcohol or sterol, and preferably n-lauryl alcohol or cholesterol; the acid-binding agent is one of triethylamine, 4-N,N-dimethylamino pyridine, pyridine, anhydrous cesium carbonate, anhydrous potassium carbonate, anhydrous sodium carbonate, sodium hydroxide and potassium hydroxide.

In the present invention, the aprotic solvent is one of dichloromethane, chloroform, acetonitrile, dimethylsulfoxide, N,N-dimethylformamide, tetrahydrofuran, 1,4-dioxane and toluene; the aprotic solvent is preferably dichloromethane or acetonitrile.

In the present invention, a molar ratio of γ-polyglutamic acid, imiquimod, liposoluble alcohol and the acid-binding agent is 10:(0.5-1.5):(1-3):(10-15); in the step (1), a mass ratio of γ-polyglutamic acid to the chlorinating agent is 1:(1-2.5), and preferably 1:1, and a reaction time is 20-25 hours.

In the present invention, a reaction temperature of the steps (1) and (2) is 10-40° C., and preferably 20-25° C.

In the present invention, in the step (1), each gram of γ-polyglutamic acid is dispersed in 5-70 mL of the aprotic solvent.

In the present invention, the preparation method further comprises a step of labeling with fluorescein; the fluorescein is amino-fluorescein or amino-rhodamine, and preferably 5-aminofluorescein.

In the present invention, the purification step comprises: removing the solvent, soaking the residual solid in anhydrous acetone, methanol, ethanol or acetonitrile, filtering, washing, and vacuum drying.

The present invention further provides use of the amphiphilic imiquimod-grafted lauryl 7-polyglutamate as a vaccine adjuvant.

In the present invention, the vaccine is a vaccine for hand-foot-and-mouth disease, avian influenza, newcastle disease, pseudorabies, porcine parvovirus, swine fever and porcine reproductive and respiratory syndrome; a mass ratio of the amphiphilic imiquimod-grafted lauryl γ-polyglutamate to an antigen is (0.01-1):(0.5-1).

In order to overcome the drawbacks of poor water solubility and large toxic and side effects of the existing R837 immunoadjuvant, the present invention uses γ-polyglutamic acid as a hydrophilic polymer skeleton, couples 5-aminofluorescein and R837 through an amide covalent bond, and couples hydrophobic liposoluble alcohol (such as n-lauryl alcohol) through an ester bond to form an amphiphilic polymer FIP (FL-γ-PGA-R837-LA). Generally, γ-Polyglutamic acid (γ-PGA) is modified in view of its carboxyl forming esters and amides upon EDC/NHS activation. Amidation coupling is difficult to carry out by using this method because the amine of imiquimod is very inactive. Therefore, the carboxyl of γ-PGA is firstly prepared into high-activity acyl chloride by thionyl chloride, oxalyl chloride or phosphorus pentachloride under the catalysis of N,N-dimethylformamide, and then the acyl chloride reacts with the amino of imiquimod to be successfully coupled through an amide bond. Through the literature search of Scifinder and Web of Science, the discovery of a method for preparing a carboxylic acid group of γ-PGA into an acyl chloride and then esterifying or amidating the acyl chloride has not been reported. The reason for this may be that the chlorination conditions are not easy to control, and the reaction system is easy to carbonize and blacken. The present invention successfully realizes the generation of acyl chloride by controlling the reaction temperature and the adding speed of the chlorinating agent. Therefore, the preparation method for FIP and γ-PGA-R837-LA disclosed herein is simple and ingenious. The FIP and γ-PGA-R837-LA in the present invention have low cost due to rich sources, good biosafety and low price of raw materials.

The FIP and γ-PGA-R837-LA prepared herein have good water solubility, good biocompatibility and also reduced toxic and side effects, and in addition, can effectively stimulate the immune response of an organism and increase the secretion level of IgG under the condition of significantly reducing the dosage of imiquimod, which can be used in the fields of vaccines, medicine loading, probes and the like. Animal experiments show that the skin of an injection site, liver, spleen and kidney of mice inoculated with the FIP-containing vaccine have no pathological changes, indicating that the mice in an experimental group have normal physical sign indexes and no adverse side effects. After different types of FIP-containing vaccines are inoculated to mice, the titer level is significantly increased, and the titer is about 2 times of that of the vaccine only containing OVA 6 weeks after first immunization, which indicates that FIP is a good water-solution immunological adjuvant with better safety, and thus is an ideal multi-type adjuvant. FIP contains fluorescent groups that can be used for in vivo/in vitro fluorescence tracking and quantification, thereby visually determining the relation between humoral immunity and cellular immunity. The FIP disclosed herein can be prepared into W, O/W, W/O and W/O/W vaccines, all of which can significantly improve the immune efficacy. Since FIP is an amphiphilic high-molecular polymer, it can form nanoparticles (micelles or vesicles) through self-assembly by intermolecular force in an aqueous or oil phase medium, and a new method is provided for the preparation of drugs and vaccine carriers. FIP (FL-γ-PGA-R837-LA) can be physically mixed with multiple antigens to prepare into a mucosal vaccine for nasal drop and oral administration.

### BRIEF DESCRIPTION OF THE DRAWINGS

FIG. 1 is a synthetic route of amphiphilic imiquimod-grafted lauryl γ-polyglutamate (γ-PGA-R837-LA);

FIG. 2 is a synthetic route of fluorescein-labeled amphiphilic imiquimod-grafted lauryl γ-polyglutamate (FIP, FL-γ-PGA-R837-LA) and a chemical structural formula of 5-aminofluorescein;

FIG. 3 is a ¹H NMR diagram of fluorescein-labeled amphiphilic imiquimod-grafted lauryl γ-polyglutamate (FL-γ-PGA-R837-LA);

FIG. 4 is a representation diagram of UV-vis of fluorescein-labeled amphiphilic imiquimod-grafted lauryl γ-polyglutamate (FL-γ-PGA-R837-LA);

FIG. 5A is a fluorescence spectrum representation diagram of FIP and imiquimod;

FIG. 5B a fluorescence spectrum representation diagram of FIP and fluorescein;

FIG. 6 is a Zeta potential diagram of fluorescein-labeled amphiphilic imiquimod-grafted lauryl γ-polyglutamate (FL-γ-PGA-R837-LA) and γ-polyglutamic acid (γ-PGA);

FIG. 7 is experimental results of survival rates of RAW 264.7 cells by fluorescein-labeled amphiphilic imiquimod-grafted lauryl γ-polyglutamate (FL-γ-PGA-R837-LA) with different concentrations, wherein the abscissa represents the concentration, the ordinate represents the cell survival rate (%), and FIP represents FL-γ-PGA-R837-LA solutions with different concentrations; IMQ represents R837 solution with different concentrations; HAc represents an acetic acid aqueous solution with a pH of 6.0; PBS represents a PBS buffer;

FIG. 8A is fluorescent flowcharts of RAW 264.7 cells phagocytosing fluorescein-labeled amphiphilic imiquimod-grafted lauryl γ-polyglutamate (FL-γ-PGA-R837-LA): an FSC-SSC scattergram of FIP interfering cells at a concentration of 50 μg/mL, with the abscissa being the intensity of the forward scattered light and the ordinate being the intensity of the side scattered light;

FIG. 8B is fluorescent flowcharts of RAW 264.7 cells phagocytosing fluorescein-labeled amphiphilic imiquimod-grafted lauryl γ-polyglutamate (FL-γ-PGA-R837-LA): a single parameter histogram of FIP interfering cells at a concentration of 50 μg/mL, with the abscissa being the value of the fluorescence signal;

FIG. 8C is fluorescent flowcharts of RAW 264.7 cells phagocytosing fluorescein-labeled amphiphilic imiquimod-grafted lauryl γ-polyglutamate (FL-γ-PGA-R837-LA): an average fluorescence diagram of FIP interfering cells with different concentrations, with the abscissa being the FIP concentration and the ordinate being the fluorescence intensity;

FIG. 9A is photographs of the skin condition of a mouse 5 days after immunization, with the area of the inoculation site within the dotted lines: injected subcutaneously with a vaccine containing imiquimod (R837, IMQ);

FIG. 9B is photographs of the skin condition of a mouse 5 days after immunization, with the area of the inoculation site within the dotted lines: injected subcutaneously with an FIP-containing vaccine;

FIG. 10 is a diagram comparing the immune effects of FIP and imiquimod (R837, IMQ) as adjuvants;

FIG. 11 is histological examination of the skin and internal organs (e.g., liver, kidney and spleen) of the injection sites of inoculated mice, wherein the first row is inoculated with PBS buffer, and the second row is inoculated with vaccine 1; a is the skin of the injection site, b is the liver, c is the spleen and d is the kidney; and

FIG. 12A is serum IgG antibody levels of mice inoculated with different types of FIP/OVA vaccines: the antibody titers two weeks after vaccination. FIP/OVA are each vaccine (containing both FIP and OVA) and OVA is each positive control vaccine (containing OVA alone);

FIG. 12B is serum IgG antibody levels of mice inoculated with different types of FIP/OVA vaccines: the serum antibody titers six weeks after vaccination. FIP/OVA are each vaccine (containing both FIP and OVA) and OVA is each positive control vaccine (containing OVA alone).

### DETAILED DESCRIPTION

The present invention will be further illustrated with reference to the following example and drawings, which, however, are not intended to limit the embodiments of the present invention.

### Example 1: Preparation and Identification of Amphiphilic Imiquimod-Grafted Lauryl γ-Polyglutamate and Fluorescein Marker Thereof

### Preparation of Amphiphilic Imiquimod-Grafted Lauryl γ-Polyglutamate (Abbreviated as 7-PGA-R837-LA)

The preparation method for γ-PGA-R837-LA comprises the following steps:
(1) dispersing 0.1 mol (13 g) of γ-polyglutamic acid (γ-PGA, MW=700,000, Xuankai Biotech Co., Ltd.) in 200 mL of anhydrous dichloromethane (DCM), adding 0.5 mL of N,N-dimethylformamide (DMF) as a catalyst, and adding dropwise 0.1 mol (7.3 mL) of thionyl chloride (SOCl₂) under stirring at room temperature (20-25° C.) at a dropwise addition rate of 2 sec/drop, with the system being operated in an anhydrous state; and after the dropwise addition was completed, reacting the system for 24 h, and absorbing the tail gas generated during the reaction by a 10% NaOH aqueous solution;
(2) adding 0.01 mol (2.4 g) of imiquimod (R837), 0.02 mol (3.72 g) of n-lauryl alcohol (LA) and 0.12 mol (12 g) of an acid-binding agent triethylamine (NEt₃) into the solution obtained in the step (1) after the reaction and reacting the system at room temperature (20-25° C.) for 48 h; and
(3) removing the solvent by rotary evaporation, soaking the residual solid in absolute methanol, filtering to obtain a filter residue, washing the filter residue with water, and vacuum drying to obtain the amphiphilic imiquimod-grafted lauryl γ-polyglutamate.

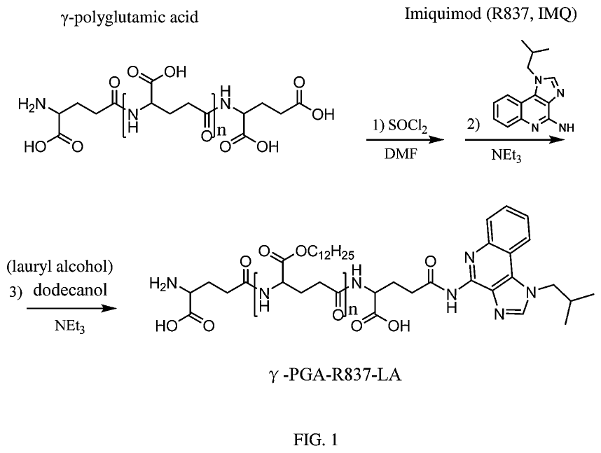

The reaction principle is shown in FIG. 1.

2. Preparation of fluorescein-labeled amphiphilic imiquimod-grafted lauryl γ-polyglutamate (abbreviated as FL-γ-PGA-R837-LA and abbreviated as FIP)

The preparation method for FIP comprises the following steps:
(1) dispersing 0.1 mol (13 g) of γ-polyglutamic acid (γ-PGA, MW=700,000, Xuankai Biotech Co., Ltd.) in 200 mL of anhydrous dichloromethane (DCM), adding 0.5 mL of N,N-dimethylformamide (DMF) as a catalyst, and adding dropwise 0.1 mol (7.3 mL) of thionyl chloride (SOCl₂) under stirring at room temperature (20-25° C.) at a dropwise addition rate of 2 sec/drop, with the system being operated in an anhydrous state; and after the dropwise addition was completed, reacting the system for 24 h, and absorbing the tail gas generated during the reaction by a 10% NaOH aqueous solution;
(2) adding 0.01 mol (2.4 g) of imiquimod (R837), 0.02 mol (3.72 g) of n-lauryl alcohol (LA), 0.5 mmol (0.17 g) of 5-aminofluorescein (abbreviated as 5-NH₂—FL) and 0.12 mol (12 g) of an acid-binding agent triethylamine (NEt₃) into the solution obtained in the step (1) after the reaction and reacting the system at room temperature (20-25° C.) for 48 h; and
(3) removing the solvent by rotary evaporation, soaking the residual solid in absolute methanol, filtering to obtain a filter residue, washing the filter residue with water, and vacuum drying to obtain the fluorescein-labeled amphiphilic imiquimod-grafted lauryl γ-polyglutamate (FL-γ-PGA-R837-LA).

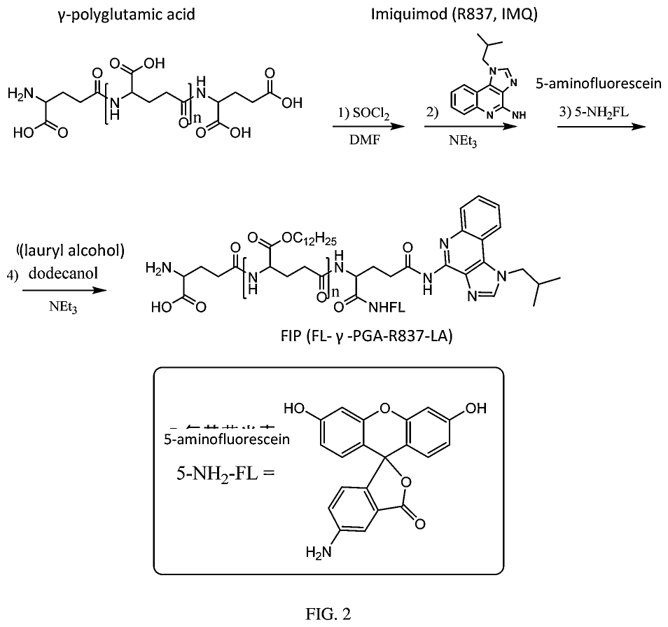

The reaction principle is shown in FIG. 2.

3. Substance identification
(1) Nuclear magnetic detection

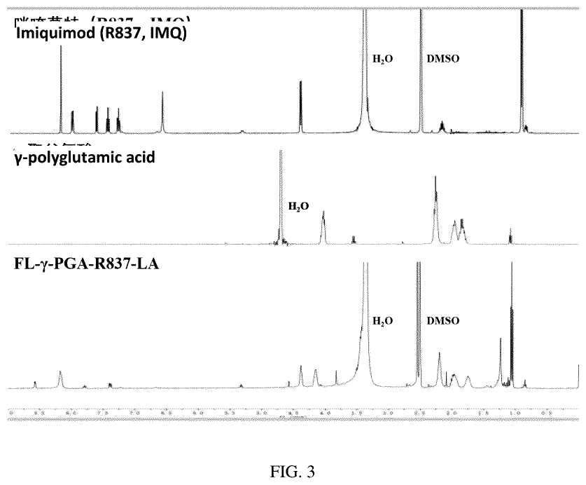

FL-γ-PGA-R837-LA was dissolved in DMSO-d₆ for representation of nuclear magnetic resonance hydrogen spectroscopy (¹H NMR). Meanwhile, γ-polyglutamic acid (dissolved in D₂O) and R837 (dissolved in DMSO-d₆) were taken as controls for evaluation of grafting and quantification of R837. The nuclear magnetic detection result is shown in FIG. 3, in the ¹H NMR spectrogram of FL-γ-PGA-R837-LA, 7-9 ppm is the chemical shift of hydrogen in an R837 aromatic-ring region, which indicates that R837 has been grafted onto a γ-polyglutamic acid skeleton, and meanwhile, the integral area ratio of R837 to γ-polyglutamic acid indicates that the grafting rate (the mass percentage content of the R837 in FL-γ-PGA-R837-LA) is about 10%; the peak appearing in the high field region indicates that the n-lauryl alcohol was grafted onto the γ-polyglutamic acid skeleton. Due to the very low content of fluorescein in FL-γ-PGA-R837-LA, the peak of FL-γ-PGA-R837-LA was very weak as affected by noise on the ¹H NMR spectroscopy.

(2) Ultraviolet-visible (UV-vis) detection

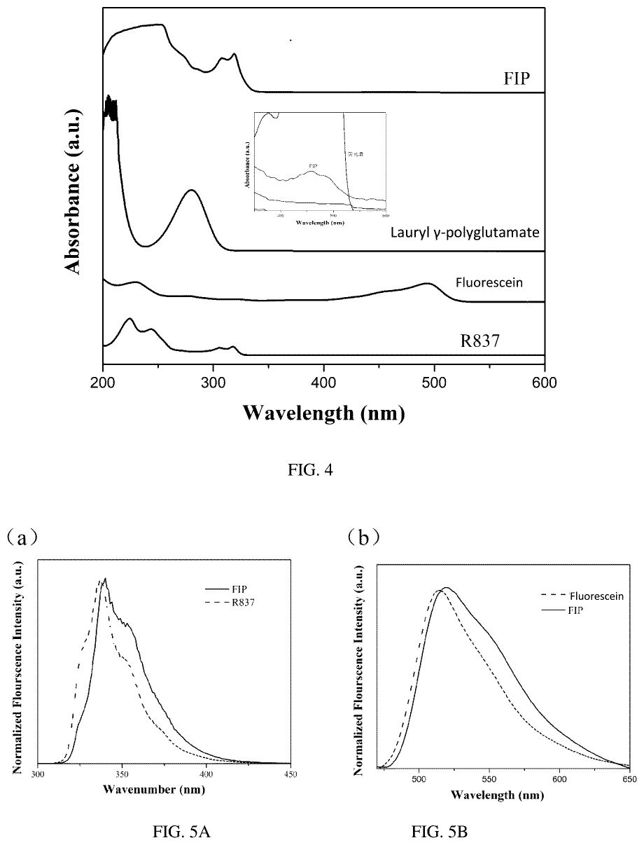

The UV-vis detection is high in sensitivity. The nuclear magnetic resonance hydrogen spectroscopy (¹H NMR) is difficult to detect due to the low content of grafted fluorescein. In order to determine whether R837 and 5-aminofluorescein were grafted, FL-γ-PGA-R837-LA was dissolved in ultrapure water, and main components in FL-γ-PGA-R837-LA were detected with UV-vis (ultraviolet-visible spectrophotometer); R837 was dissolved in ultrapure water (pH 6) acidified with hydrochloric acid as control 1; a 5-aminofluorescein aqueous solution was taken as control 2. The absorbance was measured at a wavelength of 200-600 nm, and the measurement results were normalized and then compared. The results are shown in FIG. 4, and in combination with a characteristic peak of R837 at 200-250 nm, a characteristic peak of γ-PGA at 250-300 nm and a characteristic peak of 5-FL at 475-525 nm in an ultraviolet diagram of FL-γ-PGA-R837-LA and other single components, both R837 and fluorescein were proved to be grafted onto a γ-PGA skeleton, and the structure of FL-γ-PGA-R837-LA was verified.

(3) Fluorescence (FL) detection

To further confirm whether R837 and 5-aminofluorescein were grafted onto the γ-PGA side chain, FL-γ-PGA-R837-LA was dissolved in ultrapure water and detected with a fluorescence spectrophotometer. R837 was dissolved in ultrapure water (pH 6) acidified with hydrochloric acid as control 1; a 5-aminofluorescein aqueous solution was taken as control 2. The results are shown in FIG. 5, when the R837 chromophore was excited using light having an excitation wavelength (λ_{Ex}) of 280 nm, after normalization, it was found that the R837 emission peaks (λ_{Em}=340 nm) of FL-γ-PGA-R837-LA and R837 were almost the same in peak shape with a shift of only 3 nm, indicating that R837 had been grafted onto the γ-PGA side chain; when the fluorescein chromophore was excited using light having an excitation wavelength (λ_{Ex}) of 455 nm, FL-γ-PGA-R837-LA showed a fluorescein emission peak (λ_{Em}=519 nm), and after normalization, maximum emission peaks of FL-γ-PGA-R837-LA and 5-aminofluorescein were only shifted by 4 nm, but the peak shapes of these two were almost the same, so that the 5-aminofluorescein was grafted onto the γ-PGA side chain.

(4) Zeta potential detection

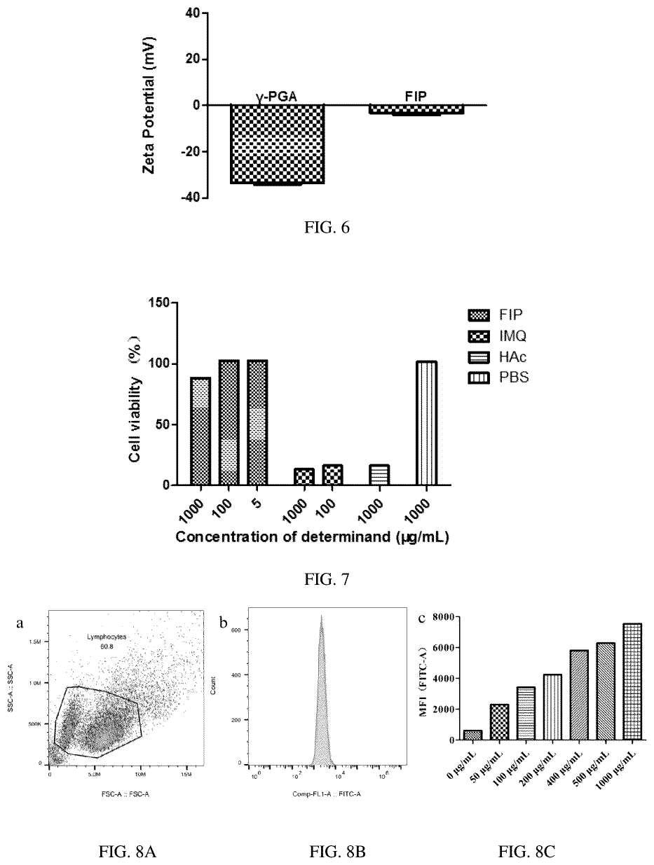

The positive and negative Zeta potential values correspond to the stability of substance structure and positive and negative charges. As can be seen from FIG. 6, the Zeta potential value of FL-γ-PGA-R837-LA is −3.3 mV, indicating that FL-γ-PGA-R837-LA can rapidly coagulate to form a gel-like substance if the concentration is too high.

### (5) Solubility Detection

R837, 7-PGA-R837-LA and FL-γ-PGA-R837-LA were dissolved in an aqueous phase (such as ultrapure water, 0.1 M PBS buffer with a pH of 7.4, 0.9% normal saline, 5% glucose aqueous solution and acidic aqueous solution with a pH of 1-6, and SBF (simulated body fluid)) and a solvent phase (ethanol, DMSO and DMF), respectively, the solubility was compared and observed, and the feasibility of a water-solution immunological adjuvant and the selection of an injection buffer were evaluated.

The results showed that 20 mg of γ-PGA-R837-LA and FL-γ-PGA-R837-LA were dissolved completely in 5 mL of ultrapure water, 0.1 M PBS buffer with a pH of 7.4, 0.9% normal saline, 5% glucose aqueous solution, acidic aqueous solution with a pH of 1-6 and SBF (simulated body fluid); 20 mg of γ-PGA-R837-LA and FL-γ-PGA-R837-LA were dissolved completely in 3 mL of DMSO, ethanol, DMF and acetone. Therefore, γ-PGA-R837-LA and FL-γ-PGA-R837-LA are amphiphilic materials and are expected to be used for preparing water-solution immunological adjuvants.

R837 cannot be dissolved in ultrapure water, 0.1 M PBS buffer with a pH of 7.4, 0.9% normal saline, 5% glucose aqueous solution and SBF (simulated body fluid), and only can be dissolved in acidic aqueous solution with a pH≤6; R837 is slightly soluble in hot DMSO and DMF solutions. Therefore, its use in water-soluble immunological adjuvants is limited.

### Example 2: In-Vitro FIP Assay and Comparison of Immune Effects Between FIP and R837

(1) In-vitro cytotoxicity assay of FIP

FIP solutions with different concentrations were prepared using a PBS buffer with a pH of 7.4 as a solvent. R837 solutions with different concentrations were prepared using an acetic acid aqueous solution with a pH of 6 as a solvent. The toxicity of FIP solution and R837 solution to cells was determined. An acetic acid aqueous solution with a pH of 6.0 was taken as a negative control, a PBS buffer with a pH of 7.4 was taken as a blank control, mouse macrophage RAW 264.7 was taken as a model source, and the toxicity was determined by using a CCK-8 method, wherein the method comprises the following steps: plating RAW 264.7 cells on a 96-well plate, incubating the cells for 24 h in a biological environment of 5% CO₂/95% 02 at 37° C., and when the cell count reached 2×10⁶ cells/well under a microscope, adding 100 μL of FIP solution or R837 solution with different concentrations into each well, incubating the mixture for 24 h, then adding a CCK-8 reagent (reagent in a cell proliferation kit, purchased from Biosep), incubating for 4 h, and determining the apoptosis degree with a microplate reader (BioTek microplate reader). As can be seen from FIG. 7, the results showed that when the concentration of FIP solution was 1000 μg/mL (containing 100 μg/mL of R837), the survival rate of the cells was maintained at 88%, and when the concentration of FIP solution was less than 1000 μg/mL, no cell apoptosis was observed, and the cell survival rate was similar to that of the cells treated by PBS buffer; in contrast, the R837 solution was more harmful to the cells, and at a concentration of 100 μg/mL, a large number of RAW 264.7 cells were apoptotic. Therefore, FIP has good biocompatibility and low toxicity, and can be used in the fields of vaccines, drug loading, probes and the like.

(2) Fluorescent flow assay for FIP/OVA pure water type

A 50 μg/mL FIP solution was prepared using a PBS buffer with a pH of 7.4 as a solvent, wherein R837 has a concentration of 5 μg/mL.

RAW 264.7 cells were plated on a 24-well cell plate and incubated for 18 h until the cell count reached 2×10⁶ cells/well, and 100 μL or 50 μg/mL of an FIP solution was added to each well. After incubation for 24 h, the cells were pipetted, washed three times with PBS, centrifuged and finally resuspended in ice PBS before assay with flow cytometer (BD FACSCalibur flow cytometer). In addition, the same method as described above was adopted to determine the effects of FIP solutions with different concentrations on RAW 264.7 cells. As can be seen from FIG. 8, the results showed that when the FIP concentration was 50 μg/mL, a distinct fluorescence peak could still be seen in RAW 264.7 cells, and undifferentiated CD³⁺ cells exhibited strong activity. With the increase of FIP concentration, the fluorescence intensity of the cells gradually increased. These results show that FIP is readily taken up by cells, can present a fluorescent marker in tissues and organs of an organism, and provide assistance in tracking immune pathways over different time periods.

(3) Comparison of immune effects between FIP and R837

1000 μg/mL of an OVA (ovalbumin) solution and 200 μg/mL of an FIP solution were prepared by taking a PBS buffer with a pH of 7.4 as a solvent, and then the two solutions were mixed in equal volumes to obtain an FIP-containing vaccine.

1000 μg/mL of an OVA solution and 200 μg/mL of an R837 solution were prepared by taking an acetic acid aqueous solution with a pH of 6 as a solvent, and then the two solutions were mixed in equal volumes to obtain an R837-containing vaccine.

In addition, 500 μg/mL of an OVA solution (abbreviated as OVA) was prepared by taking a PBS buffer with a pH of 7.4 as a solvent; 100 μg/mL of an FIP solution (abbreviated as FIP) was prepared by taking a PBS buffer with a pH of 7.4 as a solvent.

Female Balb/c mice (20-25 g) of 6-8 weeks old were randomly divided into 5 groups with 6 mice per group, and were inoculated with an FIP-containing vaccine, an R837-containing vaccine, 500 μg/mL of an OVA solution, 100 μg/mL of an FIP solution and a PBS buffer with a pH of 7.4, respectively, at a dose of 200 μL/mouse. After inoculation, the skin condition of the injection site was observed over one week. Blood was collected from the orbit 28 days after inoculation, and sera were separated and the antibody titer was measured.

The antibody titer was measured by adopting the following method: 10 μg/mL of an OVA protein solution was prepared by taking 0.05 mol/L of a Na₂CO₃—NaHCO₃ buffer with a pH of 9.6 as a solvent. A 96-well plate was coated with 50 μL of an OVA protein solution, followed by adsorption overnight at 4° C.; the solution was discarded, and the plate was washed for 2 times by using a PBST buffer (obtained by adding 500 μL of Tween-20 into 1 L of 0.1 M PBS buffer with a pH of 7.4), and placed on clean absorbent paper and patted to dryness; 100 μL of a blocking solution (obtained by adding 10 g of BSA into 1 L of 0.1 M PBS (pH 7.4) buffer) was added into each well, then the membrane was sealed, and the plate was placed in a shaker at 37° C. for incubation for 1 h, then washed 2 times by using a PBST buffer, and placed on clean absorbent paper and patted to dryness; 100 μL of mouse serum diluted with a PBST buffer was added, the plate was incubated for 1.5 h at 37° C. in the dark, the solution was discarded, and the plate was washed 5 times with a PBST buffer, and then placed on clean absorbent paper and patted to dryness; 50 μL of HRP-labeled goat anti-mouse IgG secondary antibody (purchased from Beyotime, product No. A0216) was added into each well, the plate was incubated at 37° C. in the dark for 1 h, the solution was discarded, and the plate was washed 5 times with a PBST buffer, and then placed on clean absorbent paper and patted to dryness; 50 μL of a color development solution (obtained by mixing a solution A and a solution B in the bi-component TMB color development solution purchased from InnoReagents according to a volume ratio of 1:1) was added into each well, followed by incubation for 30 min in the dark at 37° C., and finally, 50 μL of a stop solution (2 mol/L H₂SO₄ aqueous solution) was added into each well, and the absorbance was measured at 450 nm by using a microplate reader.

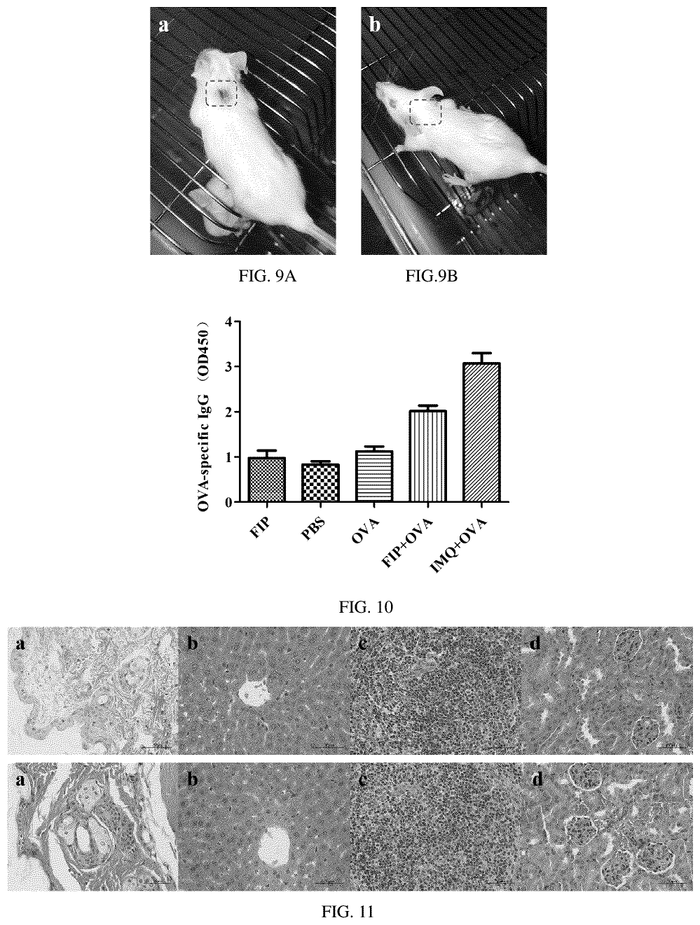

As can be seen from FIG. 9, the results showed that the mice inoculated with the R837-containing vaccine had skin fester, red swelling, suppuration and other adverse reactions in a small area; the mice inoculated with the FIP-containing vaccine had soft fur, stable diet, normal vital signs and no inflammation reaction such as red swelling and swelling at the injection site. As can be seen from FIG. 10, while imiquimod had the highest IgG titer as an adjuvant (OD₄₅₀ nm=3.07), FIP still exhibited a relatively strong titer as a water-soluble immunological adjuvant (OD₄₅₀ nm=2.1). The imiquimod component in the FIP-containing vaccine is only 10 μg/mL which is only 10% of the imiquimod component in the R837-containing vaccine, so that the FIP, serving as a water-soluble immunological adjuvant, has good water solubility and reduced toxic and side effects, and also can effectively stimulate the immune response of an organism under the condition of significantly reducing the dosage of imiquimod, and thus improves the secretion level of IgG.

Since the concentration of the imiquimod structure in the above immunization experiment was only 10 μg/mL, the mice had no inflammation reaction such as red swelling and swelling at the injection site. In order to verify whether toxic and side effects exist at a high concentration, 1000 μg/mL of an OVA solution and 1000 μg/mL of an FIP (namely FL-γ-PGA-R837-LA) solution were prepared by taking a PBS buffer with a pH of 7.4 as a solvent, and then the two solutions were mixed in equal volumes to obtain the vaccine containing 1000 μg/mL of FIP. The inoculation was carried out by adopting the same method described above, the mice had soft fur, stable diet, normal vital signs and no inflammation reaction such as red swelling and swelling at the injection site. Therefore, FIP had no toxic and side effects.

### Example 3: Immunization Experiment

This example illustrates use of the fluorescein-labeled amphiphilic imiquimod-grafted lauryl γ-polyglutamate (FL-γ-PGA-R837-LA, FIP for short) prepared in Example 1 as a vaccine immunoadjuvant.

I. Preparation of vaccine formulation

Ovalbumin (OVA) was taken as a model antigen, vaccines having different formulas were prepared as shown in Table 1, and then the mice were injected subcutaneously.

TABLE 1
Vaccine compositions and immunization dosages

| Vaccine numbering or | OVA | FIP | | Dosage for |
|---|---|---|---|---|
| composition | concentration | Concentration | Type | injection |
| 0.1M PBS buffer with pH | 0 | 0 | W | 200 μL |
| of 7.4 | | | | |
| Positive control vaccine 1 | 500 μg/mL | 0 | W | 200 μL |
| Positive control vaccine 2 | 500 μg/mL | 0 | O/W | 200 μL |
| Positive control vaccine 3 | 500 μg/mL | 0 | W/O | 200 μL |
| Positive control vaccine 4 | 500 μg/mL | 0 | W/O/W | 200 μL |
| Vaccine 1 | 500 μg/mL | 100 μg/mL | W | 200 μL |
| Vaccine 2 | 500 μg/mL | 100 μg/mL | O/W | 200 μL |
| Vaccine 3 | 500 μg/mL | 100 μg/mL | W/O | 200 μL |
| Vaccine 4 | 500 μg/mL | 100 μg/mL | W/O/W | 200 μL |

Preparation of FIP/OVA water vaccine (marked as vaccine 1): 1000 μg/mL of an OVA solution and 200 μg/mL of an FIP (namely FL-γ-PGA-R837-LA) solution were prepared by taking a 0.1 M PBS (pH 7.4) buffer as a solvent, and then the two solutions were mixed in equal volumes to obtain the FIP/OVA water vaccine (marked as vaccine 1). In vaccine 1, the FIP concentration was 100 μg/mL and the OVA concentration was 500 μg/mL. A positive control vaccine 1 was prepared according to the preparation method for vaccine 1, except that the FIP solution was replaced with water.

Preparation of FIP/OVA oil-in-water vaccine (marked as vaccine 2): Formula 4 in Chinese Patent ZL201310021011.3 was taken as an oil phase; a water phase containing OVA and FIP was prepared by taking a 0.1 M PBS (pH 7.4) buffer as a solvent; the water phase and the oil phase were mixed according to a volume ratio of 1:1, followed by homogenization under high pressure to obtain the FIP/OVA oil-in-water vaccine (marked as vaccine 2). In the vaccine, the FIP concentration was 100 μg/mL and the OVA concentration was 500 μg/mL. A positive control vaccine 2 was prepared according to the preparation method for FIP/OVA oil-in-water vaccine, except that FIP was not included in the vaccine.

Preparation of FIP/OVA water-in-oil vaccine (marked as vaccine 3): a water phase containing OVA and FIP was prepared by taking a 0.1 M PBS (pH 7.4) buffer as a solvent; the water phase and the white oil were mixed according to a volume ratio of 1:3, followed by emulsification to obtain the FIP/OVA water-in-oil vaccine (marked as vaccine 3). In vaccine 3, the OVA concentration was 500 μg/mL and the FIP concentration was 100 μg/mL. A positive control vaccine 3 was prepared according to the preparation method for vaccine 3, except that FIP was not included in the water phase.

Preparation of water-in-oil-in-water (W/O/W) vaccine: a water phase containing FIP and OVA was prepared by taking a 0.1 M PBS (pH 7.4) buffer as a solvent; the water phase and ISA201 were mixed according to a volume ratio of 1:1, followed by emulsification to obtain the FIP/OVA water-in-oil-in-water vaccine (marked as vaccine 4). In the vaccine, the FIP concentration was 100 μg/mL and the OVA concentration was 500 μg/mL. A positive control vaccine 4 was prepared according to the preparation method for vaccine 4, except that FIP was not included in the water phase.

II. Mouse immunization solution

Female Balb/c mice (20-25 g) of 6-8 week old were randomly divided into 9 groups with 6 mice per group, wherein 8 groups of mice were inoculated with positive control vaccines 1-4 and vaccines 1-4 (Table 1), respectively, and the remaining group of mice were inoculated with a 0.1 M PBS buffer with a pH of 7.4 as a negative control. The inoculation method for each vaccine was as follows: a total of two inoculations were carried out at day 1 and day 14 at a dose of 200 μL of vaccine/mouse.

III. Determination of various biochemical and immune indexes
(1) Determination of toxic and side effects of H&E staining of tissues 28 days after first immunization

Two groups of mice inoculated with a PBS buffer and vaccine 1 were subjected to slicing of the skin of an injection site and tissues of liver, kidney and spleen 28 days after first immunization so as to observe whether the mice have damage, pathological changes and other problems. The specific method was as follows: 3 mice per group were euthanized, and the skin of the injection site and internal organs (e.g., liver, kidney and spleen) were surgically separated, fixed and soaked with 4% paraformaldehyde (purchased from Leagene Biotechnology, product No. DF0135), paraffin-embedded, sliced and then subjected to immunohistochemistry staining. As can be seen from FIG. 11, the results showed that compared with the mice inoculated with the PBS buffer, the mice inoculated with vaccine 1 had no pathological changes in the skin of the injection site and liver, spleen and kidney, indicating that the mice in the experimental group have normal physical signs and no adverse side effects.

(2) Determination of IgG antibody titer

Blood was collected from the orbit every two weeks after the initial immunization, sera were separated, and specific antibody (IgG) levels in each mouse serum were determined according to the method in Example 2.

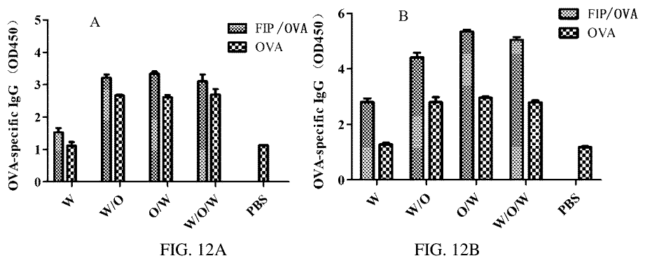

As can be seen from FIG. 12, the results showed that the FIP-containing vaccine was found to have a stronger immune response ability by comparing with different types of positive control vaccines containing OVA alone. The determination of antibody titers was carried out aiming at the type of veterinary vaccines in the market, the evaluation of the immune potency was carried out on the mice, blood was collected 2 weeks and 6 weeks after the first immunization, the antibody titers were determined by using an ELSIA method and then compared, and it could be found that O/W>W/O/W>W/O>W, indicating that the immune potency of different types of vaccines containing FIP significantly increased over the immunization time, and reached about 2 times of that of a corresponding control positive vaccine, so that the FIP is a good water-soluble immunological adjuvant with good safety, and thus is an ideal multi-type adjuvant.

In conclusion, the applicant firstly utilizes amide bonds to couple imiquimod and γ-polyglutamic acid covalent bonds, and grafts FL-γ-PGA-R837-LA polymer modified by liposoluble groups and fluorescence chromophores. FL-γ-PGA-R837-LA has good biocompatibility and amphiphilic solubility. The immune research of mice showed that the OVA was taken as a model antigen, and the γ-PGA-R837-LA or FL-γ-PGA-R837-LA was taken as an immunoadjuvant, so that the antigen-specific humoral and cellular immune response could be efficiently and persistently promoted; the entering, stimulating, transporting and metabolic processes of the vaccines can be tracked through fluorescent markers; the vaccines could be prepared into different types for use in subcutaneous injection, intramuscular injection, nasal cavity or oral administration. Therefore, a feasible solution for the design and selection of vaccine formulations is provided. Therefore, γ-PGA-R837-LA and FL-γ-PGA-R837-LA, as immunoadjuvants, have important application values in the field of immunotherapy.

What is claimed is:

1. An amphiphilic imiquimod-grafted lauryl γ-polyglutamate is prepared by a method comprising the following steps:
(i) dispersing γ-polyglutamic acid in an aprotic solvent in an anhydrous atmosphere, then adding a catalyst N,N-dimethylformamide, adding dropwise a chlorinating agent under stirring, and reacting the mixture for 10-40 hours;
(ii) adding imiquimod, liposoluble alcohol and an acid-binding agent into the solution obtained in the step (i) after reaction, and keeping reaction for 42-54 hours;
(iii) after purification, obtaining an amphiphilic imiquimod-grafted lauryl γ-polyglutamate material.

2. The amphiphilic imiquimod-grafted lauryl γ-polyglutamate according to claim 1, wherein a molecular weight of γ-polyglutamic acid is 10,000-2,000,000, and preferably 300,000-700,000; the chlorinating agent is thionyl chloride, oxalyl chloride or phosphorus pentachloride, and preferably thionyl chloride or oxalyl chloride; the liposoluble alcohol is C8-C24 alcohol, alicyclic alcohol or sterol, and preferably n-lauryl alcohol or cholesterol; the acid-binding agent is one of triethylamine, 4-N,N-dimethylamino pyridine, pyridine, anhydrous cesium carbonate, anhydrous potassium carbonate, anhydrous sodium carbonate, sodium hydroxide and potassium hydroxide.

3. The amphiphilic imiquimod-grafted lauryl γ-polyglutamate according to claim 2, wherein the aprotic solvent is one of dichloromethane, chloroform, acetonitrile, dimethylsulfoxide, N,N-dimethylformamide, tetrahydrofuran, 1,4-dioxane and toluene; the aprotic solvent is preferably dichloromethane or acetonitrile.

4. The amphiphilic imiquimod-grafted lauryl γ-polyglutamate according to claim 3, wherein a molar ratio of γ-polyglutamic acid, imiquimod, liposoluble alcohol and the acid-binding agent is 10:(0.5-1.5):(1-3):(10-15); in the step (1), a mass ratio of γ-polyglutamic acid to the chlorinating agent is 1:(1-2.5), and preferably 1:1, and a reaction time is 20-25 hours.

5. The amphiphilic imiquimod-grafted lauryl γ-polyglutamate according to claim 4, wherein a reaction temperature of the steps (1) and (2) is 10-40° C., and preferably 20-25° C.

6. The amphiphilic imiquimod-grafted lauryl γ-polyglutamate according to claim 5, wherein in the step (1), each gram of γ-polyglutamic acid is dispersed in 5-70 mL of the aprotic solvent.

7. The amphiphilic imiquimod-grafted lauryl γ-polyglutamate according to claim 6, wherein the preparation method further comprises a step of labeling with fluorescein; the fluorescein is amino-fluorescein or amino-rhodamine, and preferably 5-aminofluorescein.

8. The amphiphilic imiquimod-grafted lauryl γ-polyglutamate according to claim 7, wherein the purification step comprises: removing the solvent, soaking the residual solid in anhydrous acetone, methanol, ethanol or acetonitrile, filtering, washing, and vacuum drying.

9. A vaccine adjuvant comprising the amphiphilic imiquimod-grafted lauryl γ-polyglutamate of claim 1.

10. The vaccine adjuvant to claim 9, wherein the vaccine adjuvant is used an adjuvant in making a vaccine for hand-foot-and-mouth disease, avian influenza, newcastle disease, pseudorabies, porcine parvovirus, swine fever and porcine reproductive and respiratory syndrome; a ratio of the amphiphilic imiquimod-grafted lauryl γ-polyglutamate to an antigen is (0.01-1):(0.5-1) by weight.
